(19) United States
(12) Patent Application Publication
(10) Pub. No.: US 2016/0250234 A1
(43) Pub. Date: Sep. 1, 2016

(54) Method of Treating Endometrial Tissue Disease by Altering an Epithelial to Mesenchymal Transition

(71) Applicant: Hans M. Albertsen, Salt Lake City, UT (US)

(72) Inventor: Hans M. Albertsen, Salt Lake City, UT (US)

(21) Appl. No.: 15/141,964

(22) Filed: Apr. 29, 2016

Related U.S. Application Data
(60) Provisional application No. 62/170,346, filed on Jun. 3, 2015.

(51) Int. Cl. A61K 31/65; A61K 31/4412

(52) U.S. Cl. CPC A61K 31/65; A61K 31/4412

(57) ABSTRACT

The present invention is a method of treating endometrial tissue disease and in particular a method of treating endometriosis and adenomyosis by administering an Epithelial-to-mesenchymal transition altering compound. The compound may be a natural compound or a synthetic compound. The compound may be in pill form, liquid form, or suppository form.

### CROSS REFERENCE TO RELATED APPLICATIONS

This U.S. nonprovisional utility patent application claims the benefit under 35 USC §119(e) of U.S. provisional application No. 62/170,346 filed Jun. 3, 2015 which is incorporated herein in its entirety by this reference.

### FIELD OF THE INVENTION

The present invention relates to methods of treating endometrial tissue disease and in particular to methods of treating endometriosis and adenomyosis.

### BACKGROUND OF THE INVENTION

Endometrial tissue is tissue originating in the endometrium (see Appx A). As used herein, Endometrial Tissue Disease (ETD) shall be understood to be disease characterized by the mislocation of endometrial tissue (endometrial tissue located in other than the endometrium), and shall specifically include endometriosis and adenomyosis.

Endometriosis is a disease in which tissue that normally grows inside the uterus grows outside the uterus (see Appx B) Endometriosis may include autoimmune endometriosis, mild endometriosis, moderate endometriosis, severe endometriosis, endometriomas, superficial (peritoneal) endometriosis, deep (invasive) endometriosis, ovarian endometriosis, endometriosis-related cancers, and “endometriosis-associated conditions”. For the purpose of this invention the term endometriosis is used to describe any of these conditions.

Endometriosis is most generally defined as the presence of endometrium (glands and stroma) at sites outside of the uterus (ectopic endometrial tissues rather than eutopic or within the uterus). The most common sites are the ovaries, pelvic peritoneum, uterosacral ligaments, pouch of Douglas, and rectovaginal septum although implants have been identified on the peritoneal surfaces of the abdomen (these may grow into the intestines, ureters or bladder), in the thorax, at the umbilicus, and at incision sites of prior surgeries throughout the body (Child T J, Tan S L (2001) Endometriosis: aetiology, pathogenesis and treatment, Drugs 61:1735-1750; Giudice et al. (1998) Status of current research on endometriosis, The Journal of reproductive medicine 43:252-262), (Giudice L C (2010) Clinical practice. Endometriosis. The New England journal of medicine 362: 2389-2398.

Endometriosis is a common gynecologic disorder. The prevalence is difficult to know. It has been estimated that it affects approximately 14% of all women (range 1-43%—depending on publication and detection method), 40-60% of women with pelvic pain and 30%-50% of infertile women (Di Blasio et al. (2005) Genetics of endometriosis, Minerva ginecologica 57:225-236; Schindler A E (2004) Pathophysiology, diagnosis and treatment of endometriosis, Minerva ginecologica 56:419-435).

Adenomyosis is a medical condition characterized by the presence of ectopic tissue (glands and stroma) found (especially abnormally deep) in the uterine muscle (see Appx C).

Epithelial-to-mesenchymal transition (EMT) is a process by which epithelial cells lose their cell polarity and cell-cell adhesion, and gain migratory and invasive properties to become mesenchymal stem cells (see Appx D). Mesenchymal stem cells are multipotent stromal cells that can differentiate into a variety of cell types (see Appx E). Stromal cells are connective tissue cells derived from mesenchyme of any organ, for example in the uterine mucosa (endometrium), prostate, bone marrow, and the ovary. They are cells that support the function of the parenchymal cells of the organ (see Appx F). EMT plays an important role in organ morphogenesis during embryonic development and in wound healing (Kalluri and Weinberg (2009), The basics of epithelial-mesenchymal transition. The Journal of Clinical Investigation. 119:1420-1428; Kim et al. (2014), Role of the epithelial-mesenchymal transition and its effects on embryonic stem cells. Experimental & Molecular Medicine. 46:e108; Lamouille et al. (2014), Molecular mechanisms of epithelial-mesenchymal transition. Nature Reviews Molecular Cell Biology. 15:178-196). Pathologic effects of EMT include ETD and more recently EMT has been found to play a critical role in tumor invasion and metastasis (Loboda et al. (2011), EMT is the dominant program in human colon cancer. BMC Medical Genomics. 4:9), and in cancer stem-cell formation (Plaks et al. (2015), The cancer stem cell niche: how essential is the niche in regulating stemness of tumor cells? Cell Stem Cell. 16:225-238). Mesothelial-to-mesenchymal transition (MMT) refers to mesodermally derived epithelium (see Appx G). For the purposes of this application, unless indicated otherwise, EMT shall be considered to encompass MMT.

Several genes, previously found to be associated with endometriosis include CDC42, ID4 and VEZT (Albertsen et al. (2013), Genome-wide association study link novel loci to endometriosis. PloS one. 8:e58257; Rahmioglu et al. (2014), Genetic variants underlying risk of endometriosis: insights from meta-analysis of eight genome-wide association and replication datasets. Human Reproduction Update. 20:702-716) have been linked to EMT (Miao et al. (2013), VEZT, a novel putative tumor suppressor, suppresses the growth and tumorigenicity of gastric cancer. PloS one. 8:e74409; Rahme and Israel (2015), Id4 suppresses MMP2-mediated invasion of glioblastoma-derived cells by direct inactivation of Twist1 function. Oncogene. 34:53-62; Zhang et al. (2014), IBP regulates epithelial-to-mesenchymal transition and the motility of breast cancer cells via Rac1, RhoA and Cdc42 signaling pathways. Oncogene. 33:3374-3382). It is presented that there is a correlation between EMT and ETD and that EMT is a prerequisite for developing ETD, and in particular, EMT is a prerequisite for developing endometriosis and adenomyosis.

Various genetic markers are known to have a predictive association with ETDs. Such genetic markers and methods for use in treating endometriosis and related disease are disclosed for instance in U.S. Pat. No. 8,932,993 and U.S. patent application Ser. Nos. 12/056,754, 12/120,322, 12/566,933, 12/765,643, 13/159,132, 13/602,409, 13/603,284, 13/603,297, 13/652,018, 13/789,082, 14/594,266, 61/721,219, 61/717,048, 61/717,053, and 61/887,951, all of which are incorporated herein in their entirety by this reference.

### SUMMARY OF THE INVENTION

The present invention is a method of treating ETD, and in particular endometriosis and adenomyosis, by altering EMT in a patient having or being predisposed to ETD, and in particular endometriosis and adenomyosis. EMT of a patient is preferably altered by administering an EMT altering composition (EAC). The EAC specifically preferably affects cellular remodeling of epithelial membranes and glands that facilitate the development, implantation and or growth of endometrial tissues in a place or manner clinically characterized as ETD. More specifically the treatment is intended to inhibit or reverse EMT—a prerequisite for developing ETD.

Depending on the degree of ETD present in a patient, or the genetic and environmental risk to which a patient is exposed, a graded range of treatment is anticipated. Younger, asymptomatic patients may be treated by simple over-the-counter supplements to maintain good epithelial health and thus reduce the risk for developing ETD, whereas more severely affected symptomatic patients may be treated by compositions available only by prescription. As all individuals have a unique genetic make-up and live in unique environments the EAC formulations may benefit from optimization of the formulations and dosages in a manner dependent on genetic and environmental factors specific to a given patient. Benefits of the disclosed EAC treatment method include for instance prevention of ETD, management of ETD symptoms, regression of ETD lesions, reduction in secondary ETD complications, reduction in ETD associated pain, and reduction in ETD associated infertility. Moreover, the treatment of an ETD asymptomatic patient with an EAC to prevent ETD onset or development wherein such ETD asymptomatic patient is found to have at least one ETD associated genetic marker (including for instance Single Nucleotide Polymorphisms or SNPs, Rare Variants or RVs, and Copy Number Variants or CNVs) in the DNA of the ETD asymptomatic patient is also contemplated.

A set of markers characteristic of the epithelial state include E-cadherin (CDH1), whereas N-cadherin and Vimentin are characteristic of the mesenchymal state. EMT inhibitors include a range of known compounds that have been tested in for efficacy in tissue-culture based experiments by measuring, for example, the ratio between E-cadherin and N-cadherin. Systems like these are being used to identify novel compounds for their ability to inhibit EMT include for instance like rapamycin, 17-AAG and LY294002, and A83-01 and GDC-0941 (Chua et al. (2012), A cell-based small molecule screening method for identifying inhibitors of epithelial-mesenchymal transition in carcinoma. PloS one. 7:e33183; Reka et al. (2011), Identifying inhibitors of epithelial-mesenchymal transition by connectivity map-based systems approach. Journal of Thoracic Oncology 6:1784-1792). It is expected that the range of EAC compounds will grow and that members of this group will prove useful in the treatment of ETD via their effect on EMT.

It shall be noted that for the purposes of this application, a SNP is understood to be a genetic polymorphism having a Minor Allele Frequency (MAF) of at least 1% in a population (such as for instance the Caucasian population or the CEU population) and an RV is understood to be a genetic polymorphism having a Minor Allele Frequency (MAF) of less than 1% in a population (such as for instance the Caucasian population or the CEU population).

It shall also be noted that unless indicated otherwise, when a genetic marker (e.g. SNP or RV) is identified as the genetic marker associated with a disease (in this instance endometriosis), it shall be understood that it is the minor allele (MA) of the particular genetic marker that is associated with the disease. Further it shall also be noted that unless indicated otherwise, if the Odds Ratio (OR) of the MA is greater than 1.0, the MA of the genetic marker (in this instance the endometriosis associated genetic marker) is correlated with an increased risk of endometriosis in a case subject as compared to a control subject and shall be considered a causative marker (C), and if the OR of the MA less than 1.0, the MA of the genetic marker is correlated with a decreased risk of endometriosis in a case subject as compared to a control subject and shall be considered a protective marker (P).

It shall also be noted that unless indicated otherwise, the phrase “functional equivalent” as used herein with respect to biomarkers shall mean that a second biomarker is substantially equivalent in its diagnostic and/or prognostic value with respect to a given disease as is a first biomarker's diagnostic and/or prognostic value with respect to the given disease. A second biomarker that is in complete LD with a first biomarker shall be expressly included within the scope of “functional equivalent” with respect to the relationship between the second biomarker to the first biomarker.

### DETAILED DESCRIPTION OF THE INVENTION

The invention disclosed herein is a method of preventing or treating an ETD in a patient by administering to the patient an EAC in an amount effective to alter EMT in the patient. Included in such EMT alteration are EMT prevention, EMT inhibition, EMT reduction, and EMT reversal. Possible EACs include both natural and synthetic EACs. EACs may be specific microRNAs and protein-specific antibodies. Specific exemplary EACs include, Sorafenib, Metacycline, Diphenyl difluoroketone, Resveratrol, Curcumin, and Emodin (Dandawate et al. (2013), Novel strategies targeting cancer stem cells through phytochemicals and their analogs. Drug Delivery and Translational Research. 3:165-182; Diaz-Lopez et al. (2014), Role of microRNA in epithelial to mesenchymal transition and metastasis and clinical perspectives. Cancer Management and Research. 6:205-216; Hu et al. (2010), Curcumin inhibits transforming growth factor-beta activity via inhibition of Smad signaling in HK-2 cells. American Journal of Nephrology. 31:332-341; Ji et al. (2015), Resveratrol suppresses epithelial-to-mesenchymal transition in colorectal cancer through TGF-beta1/Smads signaling pathway mediated Snail/E-cadherin expression. BMC Cancer. 15:97; Jia et al. (2015), Sorafenib ameliorates renal fibrosis through inhibition of TGF-beta-induced epithelial-mesenchymal transition. PloS one. 10:e0117757; Li et al. (2013), Curcumin inhibits transforming growth factor-beta1-induced EMT via PPARgamma pathway, not Smad pathway in renal tubular epithelial cells. PloS one. 8:e58848; Li et al. (2013), Resveratrol inhibits the epithelial-mesenchymal transition of pancreatic cancer cells via suppression of the PI-3K/Akt/NF-kappaB pathway. Current Medicinal Chemistry. 20:4185-4194; Liu et al. (2015), miR-221 facilitates the TGFbeta1-induced epithelial-mesenchymal transition in human bladder cancer cells by targeting STMN1. BMC Urology. 15:36; Montgomery et al. (2014), MicroRNA mimicry blocks pulmonary fibrosis. EMBO Molecular Medicine. 6:1347-1356; Parvani et al. (2015), Silencing beta3 Integrin by Targeted ECO/siRNA Nanoparticles Inhibits EMT and Metastasis of Triple-Negative Breast Cancer. Cancer Research; Rico-Leo et al. (2013), Dioxin receptor expression inhibits basal and transforming growth factor beta-induced epithelial-to-mesenchymal transition. The Journal of Biological Chemistry. 288:7841-7856; Subramaniam et al. (2008), Diphenyl difluoroketone: a curcumin derivative with potent in vivo anticancer activity. Cancer Research. 68:1962-1969; Thacker and Karunagaran (2015), Curcumin and Emodin Down-Regulate TGF-beta Signaling Pathway in Human Cervical Cancer Cells. PloS one. 10:e0120045; Xi et al. (2014), Inhibition of epithelial-to-mesenchymal transition and pulmonary fibrosis by methacycline. American Journal of Respiratory Cell and Molecular Biology. 50:51-60; Zaravinos (2015), The Regulatory Role of MicroRNAs in EMT and Cancer. Journal of Oncology. 2015:865816; Zhang et al. (2015), Proton pump inhibitor pantoprazole abrogates adriamycin-resistant gastric cancer cell invasiveness via suppression of Akt/GSK-beta/beta-catenin signaling and epithelial-mesenchymal transition. Cancer Letters. 356:704-712; Zhang et al. (2015), MicroRNA-33b, upregulated by EF24, a curcumin analog, suppresses the epithelial-to-mesenchymal transition (EMT) and migratory potential of melanoma cells by targeting HMGA2. Toxicology Letters. 234:151-161). Methods of administration of such EACs include oral consumption, intravenous, subcutaneous or intra-muscular injection, intra-abdominal injection, rectal absorption, and trans-vaginal deposition. In the case of a patient manifesting symptoms of an ETD, the EAC may be for instance administered to the patient without further analysis of the patient. In the case of an ETD asymptomatic patient, the EAC may be for instance administered to the patient after having determined that the patient possess at least one RFD associated biomarker, such as at least one biomarker listed in Table 1, in the genetic material of the patient.

EACs are envisioned to be administered either as a single compound or as a combination of multiple EACs. An EAC may for instance be administered in combination with a nonsteroidal anti-inflammatory drug (NSAID) or in combination with a hormonal treatment such as an oral contraceptive (OC).

In a first exemplary embodiment of the invention, the invention comprises the step of administering to a patient known to have an ETD or to manifest symptoms of an ETD, an oral administration of an EAC of Sorafenib in pill form, resulting in the reversal of EMT in the patient and resulting in the reduction of ETD symptoms in the patient.

In a second exemplary embodiment of the invention, the invention comprises the step of administering to a patient being asymptomatic of an ETD but known to have in the genetic material of the patient a minor allele of G of rs1019142 (i.e. SEQ ID NO:001), an intra-venous injection of an EAC of Metacycline in liquid form, resulting in the inhibition of EMT in the patient and resulting in prevention of ETD symptoms in the patient.

In addition to reducing or preventing the symptoms of ETD, a benefit of the disclosed invention is that the disclosed treatment of ETD avoids hormonal and surgical methods of treatment which are the traditional methods of treating ETD. Compared to other methods of treating ETD, the disclosed treatment method has a reduced likelihood of interfering with fertility and a reduced likelihood of inducing hirsutism or other types of hormonally caused androgyny.

TABLE 1

| | | | | | | | | Case | Control | | |
|---|---|---|---|---|---|---|---|---|---|---|---|
| Set | Tbl | Name | Chr | Position | p-value | Str | OR | MAF | MAF | MA | Context Sequence |
| 001 | | rs1019142 | 16 | 7652980 | 2.27E−05 | − | 0.7562 | 0.114 | 0.1455 | G | (SEQ ID NO: 001) |
| | | | | | | | | | | | catccaaaagcagtga[C/G] |
| | | | | | | | | | | | atgaccattcaagagc |
| | | rs10869042 | 9 | 73387559 | 1.29E−05 | − | 1.287 | 0.1946 | 0.158 | C | (SEQ ID NO: 002) |
| | | | | | | | | | | | tgacatttgcattatc[C/T] |
| | | | | | | | | | | | gaggggcacactggtc |
| | | rs2650844 | 19 | 21393930 | 5.44E−07 | + | 0.7675 | 0.2018 | 0.2477 | T | (SEQ ID NO: 003) |
| | | | | | | | | | | | tgattggtgttcagca[A/T] |
| | | | | | | | | | | | actacaactttlIttg |
| | | rs6738454 | 2 | 100408730 | 3.06E−07 | + | 1.354 | 0.1961 | 0.1526 | C | (SEQ ID NO: 004) |
| | | | | | | | | | | | aaagcaagtgacgaaa[A/C] |
| | | | | | | | | | | | ctcggcgatggtctaa |
| | | rs6554072 | 4 | 53774304 | 7.53E−05 | − | 0.8346 | 0.3412 | 0.383 | C | (SEQ ID NO: 005) |
| | | | | | | | | | | | aatgaatcaagtcaga[C/G] |
| | | | | | | | | | | | gcatttttttctactc |
| | | rs10187054 | 2 | 136104943 | 1.33E−06 | − | 1.338 | 0.1797 | 0.1407 | G | (SEQ ID NO: 006) |
| | | | | | | | | | | | acaaattaatggtatc[A/G] |
| | | | | | | | | | | | ctccccaaaggacctc |
| | | rs9957245 | 18 | 15092421 | 1.87E−05 | − | 1.278 | 0.1984 | 0.1622 | T | (SEQ ID NO: 007) |
| | | | | | | | | | | | agaaacttatgcttga[C/T] |
| | | | | | | | | | | | gtgaaaccttttagga |
| | | rs2585850 | 17 | 72610080 | 6.64E−05 | + | 1.237 | 0.2005 | 0.1686 | T | (SEQ ID NO: 008) |
| | | | | | | | | | | | aggagactcatttaac[C/T] |
| | | | | | | | | | | | acttgaaagctgtata |
| | | rs2149343 | 9 | 82346568 | 5.33E−05 | − | 0.8182 | 0.247 | 0.2861 | C | (SEQ ID NO: 009) |
| | | | | | | | | | | | gatggggaaatgaaaa[C/T] |
| | | | | | | | | | | | ggatgcaatatgaaag |
| | | rs2035469 | 4 | 136508218 | 1.80E−05 | + | 1.21 | 0.443 | 0.3966 | G | (SEQ ID NO: 010) |
| | | | | | | | | | | | ctgccttctcagagtt[A/G] |
| | | | | | | | | | | | aacctgacataattta |
| 002 | | rs10111761 | 8 | 59512876 | 5.82E−06 | − | 1.371 | 0.1319 | 0.09975 | G | (SEQ ID NO: 011) |
| | | | | | | | | | | | ggatgagaattttcca[A/G] |
| | | | | | | | | | | | acttcaacgggctctg |
| | | rs157950 | 7 | 130251745 | 5.31E−05 | + | 1.383 | 0.07961 | 0.05888 | G | (SEQ ID NO: 012) |
| | | | | | | | | | | | aatgaagattcaggac[A/G] |
| | | | | | | | | | | | acgaagcagtggtaac |
| | | rs6549410 | 3 | 71963293 | 1.58E−05 | + | 1.281 | 0.1981 | 0.1617 | C | (SEQ ID NO: 013) |
| | | | | | | | | | | | agcagtcgacagatga[C/T] |
| | | | | | | | | | | | taaaactggttaccca |
| | | rs944485 | 9 | 90869638 | 2.55E−05 | − | 1.208 | 0.4191 | 0.3738 | A | (SEQ ID NO: 014) |
| | | | | | | | | | | | atgaaataacaaatgg[A/C] |
| | | | | | | | | | | | attaataacggcacaa |
| | | rs1007461 | 23 | 10257210 | 2.80E−05 | − | 1.16 | 0.4676 | 0.4308 | T | (SEQ ID NO: 015) |
| | | | | | | | | | | | caaaattcatttgagc[C/T] |
| | | | | | | | | | | | acttcacatacattgg |
| | | rs17489969 | 14 | 25712480 | 8.66E−05 | − | 1.389 | 0.0735 | 0.05404 | G | (SEQ ID NO: 016) |
| | | | | | | | | | | | aaattctccatcatga[A/G] |
| | | | | | | | | | | | gagatttcggtgcatg |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of treating ETD in a subject comprising administering an EAC to said subject such that EMT of said subject is altered.

2. The method of claim 1, wherein said ETD defines at least one of endometriosis and adenomyosis.

3. The method of claim 1, wherein said EAC defines at least one of a natural EAC and a synthetic EAC, and wherein said EAC defines an EAC in at least one of a pill form, a liquid form, and a suppository form.

4. The method of claim 1, wherein said EAC defines at least one of Sorafenib, Metacycline, Diphenyl difluoroketone, Resveratrol, Curcumin, and Emodin.

5. The method of claim 1, wherein said administering defines at least one of an oral administration, an intravenous administration, a subcutaneous administration, an intra-muscular injection administration, an intra-abdominal injection administration, a rectal absorption administration, and a trans-vaginal deposition administration.

6. The method of claim 1, wherein said subject is ETD asymptomatic.

7. The method of claim 1, wherein said subject is known to have at least one ETD associated biomarker in the genetic material of said subject.

8. The method of claim 7, wherein said at least one ETD associated biomarker defines at least one ETD associated biomarker listed in Table 1.

9. A method of treating ETD in a subject known to have at least one ETD associated biomarker in the genetic material of said subject comprising administering an EAC to said subject such that EMT of said subject is altered.

10. The method of claim 9, wherein said ETD defines at least one of endometriosis and adenomyosis.

11. The method of claim 9, wherein said EAC defines at least one of a natural EAC and a synthetic EAC, and wherein said EAC defines an EAC in at least one of a pill form, a liquid form, and a suppository form.

12. The method of claim 9, wherein said EAC defines at least one of Sorafenib, Metacycline, Diphenyl difluoroketone, Resveratrol, Curcumin, and Emodin.

13. The method of claim 9, wherein said administering defines at least one of an oral administration, an intravenous administration, a subcutaneous administration, an intra-muscular injection administration, an intra-abdominal injection administration, a rectal absorption administration, and a trans-vaginal deposition administration.

14. The method of claim 9, wherein said subject is ETD asymptomatic.

15. The method of claim 9, wherein said at least one ETD associated biomarker defines at least one ETD associated biomarker listed in Table 1.

16. A method of treating ETD in an ETD asymptomatic subject known to have at least one ETD associated biomarker in the genetic material of said subject comprising administering an EAC to said subject such that EMT of said subject is altered.

17. The method of claim 16, wherein said ETD defines at least one of endometriosis and adenomyosis.

18. The method of claim 16, wherein said EAC defines at least one of a natural EAC and a synthetic EAC, and wherein said EAC defines an EAC in at least one of a pill form, a liquid form, and a suppository form.

19. The method of claim 16, wherein said EAC defines at least one of Sorafenib, Metacycline, Diphenyl difluoroketone, Resveratrol, Curcumin, and Emodin.

20. The method of claim 16, wherein said administering defines at least one of an oral administration, an intravenous administration, a subcutaneous administration, an intra-muscular injection administration, an intra-abdominal injection administration, a rectal absorption administration, and a trans-vaginal deposition administration.

21. The method of claim 16, wherein said at least one ETD associated biomarker defines at least one ETD associated biomarker listed in Table 1.


SEQUENCE LISTING (16 sequences)

SEQ ID NO: 1 - DNA, 33 bp, Homo sapiens
catccaaaagcagtgasatgaccattcaagagc

SEQ ID NO: 2 - DNA, 33 bp, Homo sapiens
tgacatttgcattatcygaggggcacactggtc

SEQ ID NO: 3 - DNA, 33 bp, Homo sapiens
tgattggtgttcagcawactacaactttttttg

SEQ ID NO: 4 - DNA, 33 bp, Homo sapiens
aaagcaagtgacgaaamctcggcgatggtctaa

SEQ ID NO: 5 - DNA, 33 bp, Homo sapiens
aatgaatcaagtcagasgcatttttttctactc

SEQ ID NO: 6 - DNA, 33 bp, Homo sapiens
acaaattaatggtatcrctccccaaaggacctc

SEQ ID NO: 7 - DNA, 33 bp, Homo sapiens
agaaacttatgcttgaygtgaaaccttttagga

SEQ ID NO: 8 - DNA, 33 bp, Homo sapiens
aggagactcatttaacyacttgaaagctgtata

SEQ ID NO: 9 - DNA, 33 bp, Homo sapiens
gatggggaaatgaaaayggatgcaatatgaaag

SEQ ID NO: 10 - DNA, 33 bp, Homo sapiens
ctgccttctcagagttraacctgacataattta

SEQ ID NO: 11 - DNA, 33 bp, Homo sapiens
ggatgagaattttccaracttcaacgggctctg

SEQ ID NO: 12 - DNA, 33 bp, Homo sapiens
aatgaagattcaggacracgaagcagtggtaac

SEQ ID NO: 13 - DNA, 33 bp, Homo sapiens
agcagtcgacagatgaytaaaactggttaccca

SEQ ID NO: 14 - DNA, 33 bp, Homo sapiens
atgaaataacaaatggmattaataacggcacaa

SEQ ID NO: 15 - DNA, 33 bp, Homo sapiens
caaaattcatttgagcyacttcacatacattgg

SEQ ID NO: 16 - DNA, 33 bp, Homo sapiens
aaattctccatcatgargagatttcggtgcatg